(19) United States
(12) Patent Application Publication
(10) Pub. No.: US 2024/0062039 A1
(43) Pub. Date: Feb. 22, 2024

(54) Design method of polymeric seawater desalination membrane based on graph neural network

(71) Applicants: Hangzhou Dianzi University, Hangzhou City (CN); Hangzhou Dianzi University Shangyu Institute, Shaoxing City (CN)

(72) Inventors: Lijun LIANG, Hangzhou City (CN); Zhe KONG, Hangzhou City (CN); Tianyu LI, Hangzhou City (CN); Xuanchao MA, Hangzhou City (CN)

(21) Appl. No.: 18/452,525

(22) Filed: Aug. 19, 2023

(30) Foreign Application Priority Data
Aug. 19, 2022 (CN) 202210997162.1

(51) Int. Cl. G06N 3/04; G06N 3/08

(52) U.S. Cl. CPC G06N 3/04; G06N 3/08; B01D 71/06

(57) ABSTRACT

The present disclosure discloses a design method of a polymeric seawater desalination membrane based on a graph neural network, including the following steps: S1: constructing a knowledge graph of element-molecular group-molecular structure based on chemical knowledge, and constructing a molecular structure information data set; S2: obtaining related data of design and manufacture of a polymeric desalination membrane, establishing a seawater desalination membrane database, and performing characterization processing suitable for a graph neural network model with reference to analysis of the structure information data set to generate an adjacency matrix; S3: building the graph neural network; S4: outputting a design model of the polymeric seawater desalination membrane by training the graph neural network; and S5: predicting a chemical structure meeting seawater desalination membrane performance requirements for seawater desalination by using the design model of the polymeric seawater desalination membrane.

### CROSS REFERENCE TO RELATED APPLICATION

This patent application claims the benefit and priority of Chinese Patent Application No. 202210997162.1, filed with the China National Intellectual Property Administration on Aug. 19, 2022, the disclosure of which is incorporated by reference herein in its entirety as part of the present application.

### TECHNICAL FIELD

The present disclosure relates to the technical field of seawater desalination, and in particular, to a design method of a polymeric seawater desalination membrane based on a graph neural network.

### BACKGROUND

71 percent of the Earth's surface is covered by oceans, and seawater resources are precious wealth brought to us by nature. With the advancement of economic globalization, ocean trade is booming, and coastal areas, as key areas of ocean trade, are often regions with relatively developed local economy. However, although many coastal areas are economically developed, fresh water resources are quite scarce, which cannot meet the needs of people's daily life and industrial production. Salt with a high concentration in seawater seriously harms human health if the seawater is directly drunk, and the unpurified seawater further contains a lot of harmful substances. In addition, the salt with a high concentration also causes great harm to industrial devices, corrodes metals of machines, seriously affects service lives of the machines, and further causes major safety hazards. Seawater desalination technology has solved the problem of shortage of fresh water resources to some extent, but also has many shortcomings.

Currently, there are two main methods for seawater desalination, namely a distillation method and a reverse osmosis method. The distillation method is mainly used in super-large-scale seawater desalination and places rich in heat energy. A reverse osmosis membrane method is more widely used because of its very wide application range and very high desalination rate, which provides a lot of convenience for regions lacking fresh water. Currently, seawater desalination devices of various specifications emerge one after another, but people are also worried about production costs and design costs of seawater desalination. To reduce energy consumption of seawater desalination, scientists are constantly studying the design direction of seawater desalination membranes. However, since the breakthrough of composite polyamide membranes in terms of seawater desalination nearly half a century ago, there is only slight improvement in water-salt selectivity of seawater desalination membranes in recent years. A large part of the reason for the slow progress is that design verification of conventional seawater desalination membranes based on experimental data is very high in cost and has high requirements for experimenters and devices, the scheme verification needs a long period, and the understanding of membrane synthesis-structure-performance is limited, which is not conducive to large-scale development.

In recent years, the rise and application of a neural network has successfully promoted the development of digitalization in conventional industries. Many methods that once relied heavily on manual feature extraction and inductive research have now been replaced by various deep learning methods. However, in the design field of polymeric seawater desalination membranes, it is difficult to use a conventional neural network to fully express a complex data structure of a polymer, so that it is difficult to train the network. Existing design methods cannot effectively use global information of molecules, and cannot model overall characteristics of data in terms of structure and function, so that accuracy and reliability of prediction need to be improved.

### SUMMARY

In view of the shortcomings of the prior art that the existing neural network cannot well describe molecular information. To address the high design cost and time-consuming characteristic, the present disclosure provides a design method of a polymeric seawater desalination membrane based on a graph neural network The technical solution of the present disclosure is as follows:

A design method of a polymeric seawater desalination membrane based on a graph neural network includes the following steps:
S1: constructing a knowledge graph of element-molecular group-molecular structure based on chemical knowledge, and constructing a molecular structure information data set, where data in the molecular structure information data set includes atoms in a molecule, related chemical bonds connected, and characteristics of the molecule;
S2: obtaining related data of design and manufacture of a polymeric desalination membrane from literature and other databases, establishing a seawater desalination membrane database, and performing characterization processing suitable for a graph neural network with reference to analysis of the structure information data set to generate an adjacency matrix, where the related data of the seawater desalination membrane database includes a test temperature, seawater desalination performance and a molecular structural formula of the seawater desalination membrane;
S3: building the graph neural network, where atoms of the desalination membrane polymer are used as nodes of the graph, molecular bonds are used as edges, and global nodes are added to generate the graph neural network; and initializing the graph neural network to obtain an initial input of the graph neural network, where the global nodes include overall information of a desalination membrane polymer monomer;
S4: outputting a design model of the polymeric seawater desalination membrane by training the graph neural network; and
S5: predicting a chemical structure meeting seawater desalination membrane performance requirements for seawater desalination by using the design model of the polymeric seawater desalination membrane.

Preferably, in step S1, data of the knowledge graph is screened and cleaned before the molecular structure information data set is constructed, the data of the knowledge graph includes electronegativity and oxidation of molecules and groups, and synthesis difficulty and valence electrons of compounds, and a method for the data screening and cleaning of the knowledge graph includes: matching in the literature and the database by using efficient string search and replacement algorithm flashtext, screening related data, and cleaning data with obvious errors and problems with reference to related chemical characteristics and a comparison with data of the same type.

Preferably, in step S2, a method for the characterization processing for generating the adjacency matrix includes: transforming the obtained molecular structural formula into a simplified molecular input line entry specification (SMILES) by using chemdraw software, and then obtaining the adjacency matrix of the molecule by using a GetAdjacencyMatrix method of an rdkit library.

Preferably, in step S3, when the atoms are used as nodes of the graph, a serial number of each atom, a number and serial numbers of atoms adjacent to the atom, and related attributes of the atom are input, so that the atom is used as a node; when the molecular bonds are used as edges, a serial number and weight of each molecular bond and related attributes of the molecular bond are input, so that the molecular bond is used as an edge; and in addition, attributes, that is, a number of nodes and a longest path of the graph, are added to additional molecular information provided by the global nodes.

Preferably, a method for the initialization of the graph neural network in step S3 includes:
when a main architecture of the graph neural network is initialized, generating an embedding layer of nodes based on neighbor nodes, where the embedding layer is in the following form:

$$h {v}^{k} = σ ( Wk {\sum }_{u ∈ N ( v )} \frac{h {u}^{k - 1}}{\vert N ( v ) \vert} + B k {h}_{v}^{k - 1} ) ,$$

where v represents an index of an atomic node in the graph, k represents a number of current convolutional layers, N(v) represents all neighbors of the node v, σ is an activation function LeakyReLU, W and B are model parameters to be learned, edges have independent parameters and similar operations are performed, and h represents a weight and deviation of each node in this layer of the graph neural network;
providing a fully connected layer after the embedding layer to output a result; and
initializing a weight and deviation of the graph neural network to obtain an initial input of the graph neural network:

hᵥ⁰=Xᵥ,
where X represents an input matrix of this layer of the graph neural network, including related attributes of the atoms of the desalination membrane polymer.

Preferably, the training of the graph neural network in step S4 includes the following substeps:
S4-1: performing normalization and data enhancement processing on initial input molecular information input by the graph neural network to make the initial input molecular information easy to converge and improve accuracy;
S4-2: constructing an embedding layer with the graph neural network as a main body, calculating a linear combination value of an input and a weight for each layer, and then applying the activation function to the linear combination value, which has different model parameters for nodes and edges;
S4-3: performing aggregation operation on nodes and edges in each layer, and aggregating weights of neighbor nodes and global nodes into nodes; and
S4-4: finally training by using the fully connected layer to calculate a loss function, and outputting the design model of the polymeric seawater desalination membrane.

Preferably, a standard for evaluating seawater desalination membrane samples by using the design model of the polymeric seawater desalination membrane output in step S4-4, that is, a salt rejection rate, has the following expression:

salt rejection rate=1−(the number of sodium ion+chloride ion passed)/(the number of total sodium ion+chloride ion).

Preferably, the design model of the polymeric seawater desalination membrane uses the graph neural network to learn graph structure data, extract and explore features and patterns in a molecular structure graph, and integrate graph convolution and an attention mechanism.

The present disclosure further provides a computer device, including a memory, a processor, and a program stored in the processor and executable on the processor, where when the processor executes the program, the design method of a polymeric seawater desalination membrane based on a graph neural network described above is implemented.

The present disclosure further provides a computer-readable storage medium, which stores a computer program, where when the computer program is executed by a processor, the design method of a polymeric seawater desalination membrane based on a graph neural network described above is implemented.

The present disclosure has the following characteristics and beneficial effects:

With the above technical solution, with the goal of maximizing a salt rejection rate of a desalination membrane and a ratio of water to salt passed, experimental and literature data of different desalination membranes are obtained, and a professional database of desalination membranes is established with reference to an existing database; a molecular structural formula of the seawater desalination membrane is transformed graphically to obtain a data structure for the graph neural network to be trained; an improved graph neural network algorithm is used to train the data to obtain the most suitable molecular structural formula of the polymer membrane material; and with the goal of optimizing efficiency of seawater desalination, comprehensively considering costs of raw materials, a reasonable membrane structure is selected to improve practical usability of the model. Comprehensively considering the material, shape, area and other conditions of the polymer membrane, a group of materials with superior theoretical properties are found by training by using the graph neural network model, and then experimental verification is performed. According to the present disclosure, a novel graph construction method is adopted for the molecular structural formula of the material, so that information loss can be effectively reduced. In addition, adding global nodes can effectively retain global information of the molecular structural formula and improve accuracy of prediction. Moreover, the method of the present disclosure has high reliability, good accuracy, and is scientific and effective. According to the present disclosure, a threshold of design and manufacture of the seawater desalination membrane is obviously lowered, the process of design and manufacture is accelerated, and the database can be used for development of similar products and has good reusability and broad application prospects in the field of seawater desalination membrane design.

### BRIEF DESCRIPTION OF THE DRAWINGS

To describe technical solutions in embodiments of the present disclosure or in the prior art more clearly, accompanying drawings required in the description of the embodiments or the prior art are briefly described below. Obviously, the accompanying drawings in the following description illustrate only some of the embodiments of the present disclosure, and a person of ordinary skill in the art can further obtain other drawings based on these accompanying drawings without creative efforts.

FIG. 1 shows main modules of a design method of a polymeric seawater desalination membrane based on a graph neural network according to an embodiment of the present disclosure; and

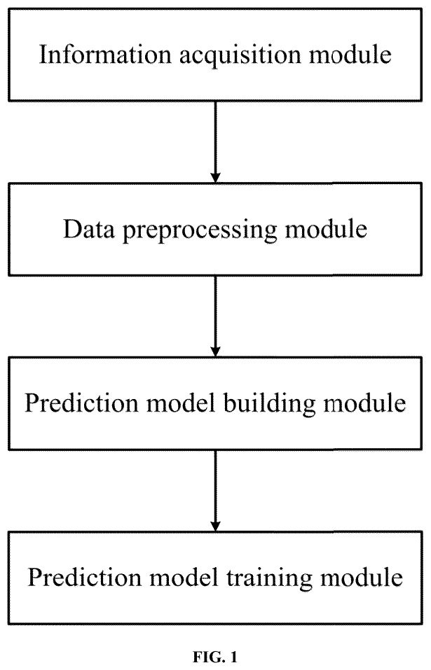

FIG. 2 shows a structure of training modules of a polymeric seawater desalination membrane design prediction model based on a graph neural network according to an embodiment of the present disclosure.

### DETAILED DESCRIPTION OF THE EMBODIMENTS

To clearly explain the advantages of the present disclosure, implementations of the present disclosure will be described in detail below with reference to examples. It should be understood that the specific implementations described herein are merely intended to explain the present disclosure but not to limit the present disclosure.

It should be understood by a person skilled in the art that, unless otherwise specified, all terms used herein (including technical terms and scientific terms) have the same meanings as those generally understood by a person of ordinary skill in the art to which the present disclosure belongs.

It should be understood by those skilled in the art that unless otherwise specified, the terms such as “first” and “second” in the description, claims, and the foregoing accompanying drawings of the present disclosure are used to distinguish between similar objects and are not necessarily used to describe a specific order or sequence. It should be understood that data used in such a way may be interchanged under appropriate circumstances so that the embodiments of the present disclosure described herein can be implemented in an order other than those illustrated or described herein. In addition, the terms “including” and “having”, and any variations thereof, are intended to cover non-exclusive inclusion, for example, a process, method, system, product, or device including a series of steps or units is not necessarily limited to those steps or units explicitly listed, but may include other steps or units not explicitly listed or inherent to these processes, methods, products, or devices.

To facilitate the understanding of the present disclosure, the present disclosure will be described in detail below with reference to the accompanying drawings.

The present disclosure provides a design method of a polymeric seawater desalination membrane based on a graph neural network, including the following steps.
S1: Construct a knowledge graph of element-molecular group-molecular structure based on chemical knowledge, and construct a molecular structure information data set, where data in the molecular structure information data set includes atoms in a molecule, related chemical bonds connected, and characteristics of the molecule; data of the knowledge graph is screened and cleaned before the molecular structure information data set is constructed, the data of the knowledge graph includes electronegativity and oxidation of molecules and groups, and synthesis difficulty and valence electrons of compounds, and a method for the data screening and cleaning of the knowledge graph includes: matching in the literature and the database by using efficient string search and replacement algorithm flashtext, screening related data, and cleaning data with obvious errors and problems with reference to related chemical characteristics and a comparison with data of the same type.
S2: Obtain related data of design and manufacture of a polymeric desalination membrane from literature and other databases, establish a seawater desalination membrane database, and perform characterization processing suitable for a graph neural network with reference to analysis of the structure information data set to generate an adjacency matrix, where the related data of the seawater desalination membrane database includes a test temperature, seawater desalination performance and a molecular structural formula of the seawater desalination membrane.

Further, a method for the characterization processing for generating the adjacency matrix includes: using an rdkit library supported by python to first convert a chemical structure SMILES into a Mol object, then generating a ConvMol object by WeaveFeaturizer and MolGraphConvFeaturizer converters of a deepchem library, then extracting the adjacency matrix and other information from the ConvMol object, transforming the obtained molecular structural formula into a simplified molecular input line entry specification (SMILES) by using chemdraw software, and then obtaining the adjacency matrix of the molecule by using a GetAdjacencyMatrix method of the rdkit library.
S3: Build the graph neural network, where atoms of the desalination membrane polymer are used as nodes of the graph, molecular bonds are used as edges, and global nodes are added to generate the graph neural network; and initialize the graph neural network to obtain an initial input of the graph neural network, where the global nodes include overall information of a desalination membrane polymer monomer, and specific overall information of the desalination membrane polymer monomer refers to aromaticity, number of atoms, maximum chain length, and other information.

Further, when the atoms are used as nodes of the graph, a serial number of each atom, a number and serial numbers of atoms adjacent to the atom, and related attributes of the atom are input, so that the atom is used as a node; when the molecular bonds are used as edges, a serial number and weight of each molecular bond and related attributes of the molecular bond are input, so that the molecular bond is used as an edge; and in addition, a number of nodes and a longest path and other attributes of the graph are added to additional molecular information provided by the global nodes, thereby adding global information to be helpful to the training of the graph neural network.

It can be understood that the overall structure of the graph neural network includes a multi-layer network structure of an input layer, an embedding layer, a hidden layer and an output layer, each layer takes multiple influencing factors into account, and each layer is composed of a plurality of neurons.

The hidden layer is a two-layer multi-layer perception (MLP) fully connected layer, and the output layer is a single neuron. Long short-term memory (LSTM) or max-pooling may also be selected for an aggregation function, so as to aggregate neighbor node information.

Specifically, when a main architecture of the graph neural network is initialized, the embedding layer of nodes is generated based on neighbor nodes, and the embedding layer is in the following form:

$$h {v}^{k} = σ ( Wk {\sum }_{u ∈ N ( v )} \frac{h {u}^{k - 1}}{\vert N ( v ) \vert} + B k {h}_{v}^{k - 1} )$$

where v represents an index of an atomic node in the graph, k represents a number of current convolutional layers, N(v) represents all neighbors of the node v, σ is an activation function LeakyReLU, W and B are model parameters to be learned, edges have independent parameters and similar operations are performed, and h represents a weight and deviation of each node in this layer of the graph neural network;
a fully connected layer is provided after the embedding layer to output a result; and
a weight and deviation of the graph neural network are initialized to obtain an initial input of the graph neural network:

hᵥ⁰=Xᵥ,
where X represents an input matrix of this layer of the graph neural network, including related attributes of the atoms of the desalination membrane polymer.
S4: Train the graph neural network to train weights of variables of the polymeric seawater desalination membrane to minimize a loss function, and output a design model of the polymeric seawater desalination membrane.

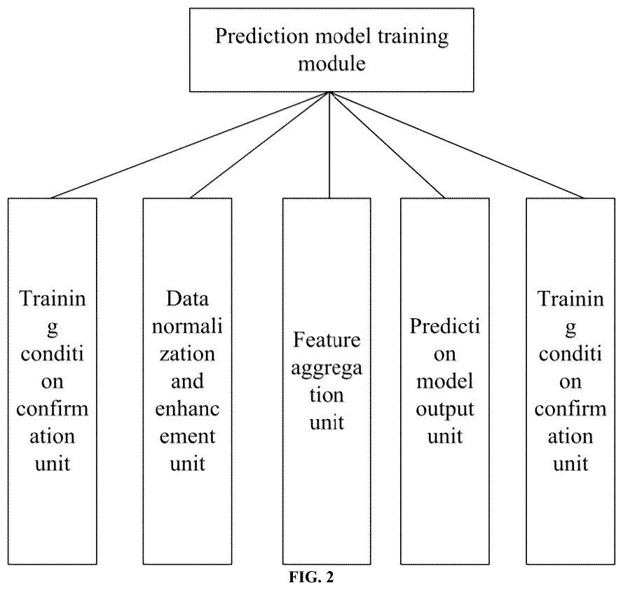

In this embodiment, as shown in FIG. 2, there are mainly five units, and a training condition confirmation unit is configured to set an objective function and constraint conditions, and determine training end conditions and target requirements of model training; and

training epoch of the graph neural network is set as 500, the loss function is a mean square error, and when a correlation coefficient of a test set is greater than 0.95 or epoch runs out, training is stopped.

Further, the training of the graph neural network in step S4 includes the following substeps.
S4-1: Perform, by a data normalization and enhancement unit, normalization and data enhancement processing on initial input molecular information input by the graph neural network to make the initial input molecular information easy to converge and improve accuracy. A matrix composed of structural characteristics of different polymers and seawater desalination performance labels is input.

Specifically, the input seawater desalination membrane database is randomly divided into an 80% training set and a 20% test set, and a mean x̅ₖ̅ and a standard deviation Sₖ of sample parameters are calculated.

$$\overline{{x}_{k}} = \frac{1}{n} {\sum }_{i = 1}^{n} {x}_{ik} , {S}_{k} = \sqrt{\frac{1}{n} {\sum }_{i = 1}^{n} {( {x}_{ik} - \overline{{x}_{k}} )}^{2}}$$

The mean and standard deviation are changed based on the following rules.

$${x}_{ik}^{″} = \frac{{x}_{ik} - \overline{{x}_{k}}}{{S}_{k}}$$

Influence parameters are normalized to the range of [0,1] by translation transformation.

$${x}_{ik}^{′} = \frac{{x}_{ik}^{″} - min ( {x}_{ik}^{″} , i ∈ [ 1 , n ] )}{max ( {x}_{ik}^{″} , i ∈ [ 1 , n ] ) - min ( {x}_{ik}^{″} , i ∈ [ 1 , n ] )}$$

Random disturbance is added to the training set to enhance the anti-interference ability of the model.
S4-2: Construct an embedding layer with the graph neural network as a main body, calculate a linear combination value of an input and a weight for each layer, and then apply the activation function to the linear combination value, which has different model parameters for nodes and edges.

It can be understood that, the embedding layer transforms a sparse matrix into a dense matrix through some linear transformations and convolution.
S4-3: Aggregate, by a feature aggregation unit, parameter features of adjacent nodes or edges, perform aggregation operation on nodes and edges in each layer, and aggregate weights of neighbor nodes and global nodes into nodes, where a core aggregation method is:

$$A G G = {\sum }_{u ∈ N ( v )} \frac{h {u}^{k - 1}}{\vert N ( v ) \vert} .$$

In this embodiment, graph convolution and an attention mechanism are integrated. When the representation of each atomic node in the graph is calculated, different weights will be assigned to the atomic nodes based on characteristics of neighbor atomic nodes. The importance is as follows:

eᵢⱼ=a(W\vec{h}ᵢ,W\vec{h}ⱼ) αᵢⱼβsoft maxⱼ(eᵢⱼ),
where a is realized by using a single-layer feedforward neural network, and the calculation method is as follows:

$${α}_{ij} = \frac{exp ( Leaky Re LU ( {\vec{a}}^{T} [ W {\vec{h}}_{i} ‖ W {\vec{h}}_{j} ] ) )}{{\sum }_{k ∈ {N}_{i}} exp ( L e aky Re LU ( {\vec{a}}^{T} [ W {\vec{h}}_{i} ‖ W {\vec{h}}_{k} ] ) )} .$$

After the attention is calculated, a new representation that a node aggregates information of its neighbor nodes can be obtained.

$${\vec{h}}_{i}^{′} = σ ( {\sum }_{j ∈ {N}_{i}} {α}_{ij} W {\vec{h}}_{j} )$$

Results of K independent calculations are combined to improve the fitting ability of the model.

$${{\vec{h}}_{i}^{′} = ‖}_{k = 1}^{K} σ ( {\sum }_{j ∈ {N}_{i}} {α}_{ij}^{k} {W}^{k} {\vec{h}}_{j} )$$

S4-4: Finally train by using the fully connected layer to calculate the loss function; when the loss function of a training result meets requirements of a training target by a prediction model output unit, end the training, perform five-fold cross-validation, and output the design model of the polymeric seawater desalination membrane.

A standard for evaluating seawater desalination membrane samples by using the design model of the polymeric seawater desalination membrane output, that is, a salt rejection rate, has the following expression:

salt rejection rate=1−(the number of sodium ion+chloride ion passed)/(the number of total sodium ion+chloride ion).

It should be noted that step S4 further includes substep S4-5, in which a retraining unit is configured to adjust, when the result does not meet training target requirements or exceeds set rounds, parameter variables to retrain until the training target requirements are met, and if the training target requirements are still not met for many times, return to step S3 for redesign.

It can be understood that, the design model of the polymeric seawater desalination membrane uses the graph neural network to learn graph structure data, extract and explore features and patterns in a molecular structure graph, and integrate graph convolution and an attention mechanism.
S5: Predict a chemical structure meeting seawater desalination membrane performance requirements for seawater desalination by using the design model of the polymeric seawater desalination membrane.

Embodiment 2 of the present disclosure provides an information data processing terminal for implementing a design of a polymeric seawater desalination membrane based on a graph neural network, including a memory and a processor that can communicate with each other.

Embodiment 3 of the present disclosure provides a computer-readable storage medium. A computer program stored in the storage medium includes instructions, which cause a design method of a polymeric seawater desalination membrane based on a graph neural network to be performed when the computer program runs on a computer. The present disclosure provides a computer-readable storage medium. A computer program stored in the computer-readable storage medium includes instructions, which enable a design method of a polymeric seawater desalination membrane based on a graph neural network to be performed when the computer program runs on a computer.

A person skilled in the art should understand that an embodiment of the present disclosure may be provided as a method, a system, or a computer program product. Therefore, the present disclosure may be in the form of a full-hardware embodiment, a full-software embodiment, or an embodiment combining software and hardware aspects. In addition, the present disclosure may be further in the form of a computer program product implemented on one or more computer-usable storage media (including, but not limited to, a disk storage, a compact disc-read-only memory (CD-ROM), and an optical memory) containing computer-usable program code.

According to embodiments of applications, the applications are described with reference to a method, a flowchart and/or a block diagram of a device (system) and a computer program product. Computer program instructions should understand each process and/or block in the flow and/or block diagram. These computer program instructions can be provided to a general-purpose computer, a special-purpose computer, an embedded processor or other programmable data processing devices to generate such machine instructions, which are executed by a processor of a computer or other programmable data processing devices and production devices to implement a flowchart or a plurality of processes and/or a block or block diagram specified in a function.

These computer program instructions can also be stored in a computer-readable memory. The memory can guide a computer or other programmable data processing devices to operate in a specific way, so that the instructions stored in the computer-readable memory are executed by a manufacturing product including an instruction device achieving the function, to perform the steps for implementing functions specified in one or more processes of the flowchart and/or one or more block diagrams.

These computer program instructions can also be loaded on a computer or other programmable data processing devices, so that a series of operation steps are performed on the computer or other programmable devices to perform computer-implemented processing, and therefore the instructions executed on the computer or other programmable devices are used to provide the steps for implementing functions specified in one or more processes of the flowchart and/or one or more block diagrams.

The specific embodiments described herein are only used to explain the present disclosure, and are not intended to define the present disclosure. Various changes may be made to the present disclosure for a person skilled in the art. Any modification or composition made by a person skilled in the art based on the spirit and principle of the present disclosure without creative efforts falls within the protection scope of the present disclosure.

What is claimed is:

1. A design method of a polymeric seawater desalination membrane based on a graph neural network, comprising the following steps:
S1: constructing a knowledge graph of element-molecular group-molecular structure based on chemical knowledge, and constructing a molecular structure information data set, wherein data in the molecular structure information data set comprises atoms in a molecule, related chemical bonds connected, and characteristics of the molecule;
S2: obtaining related data of design and manufacture of a polymeric desalination membrane from literature and other databases, establishing a seawater desalination membrane database, and performing characterization processing suitable for a graph neural network with reference to analysis of the structure information data set to generate an adjacency matrix, wherein the related data of the seawater desalination membrane database comprises a test temperature, seawater desalination performance and a molecular structural formula of the seawater desalination membrane;
S3: building the graph neural network, wherein atoms of the desalination membrane polymer are used as nodes of the graph, molecular bonds are used as edges, and global nodes are added to generate the graph neural network; and initializing the graph neural network to obtain an initial input of the graph neural network, wherein the global nodes comprise overall information of a desalination membrane polymer monomer;
S4: outputting a design model of the polymeric seawater desalination membrane by training the graph neural network; and
S5: predicting a chemical structure meeting seawater desalination membrane performance requirements for seawater desalination by using the design model of the polymeric seawater desalination membrane.

2. The design method of a polymeric seawater desalination membrane based on a graph neural network according to claim 1, wherein in step S1, data of the knowledge graph is screened and cleaned before the molecular structure information data set is constructed, the data of the knowledge graph comprises electronegativity and oxidation of molecules and groups, and synthesis difficulty and valence electrons of compounds, and a method for the data screening and cleaning of the knowledge graph comprises: matching in the literature and the database by using efficient string search and replacement algorithm flashtext, screening related data, and cleaning data with obvious errors and problems with reference to related chemical characteristics and a comparison with data of the same type.

3. The design method of a polymeric seawater desalination membrane based on a graph neural network according to claim 1, wherein in step S2, a method for the characterization processing for generating the adjacency matrix comprises: transforming the obtained molecular structural formula into a simplified molecular input line entry specification (SMILES) by using chemdraw software, and then obtaining the adjacency matrix of the molecule by using a GetAdjacencyMatrix method of an rdkit library.

4. The design method of a polymeric seawater desalination membrane based on a graph neural network according to claim 1, wherein in step S3, when the atoms are used as nodes of the graph, a serial number of each atom, a number and serial numbers of atoms adjacent to the atom, and related attributes of the atom are input, so that the atom is used as a node; when the molecular bonds are used as edges, a serial number and weight of each molecular bond and related attributes of the molecular bond are input, so that the molecular bond is used as an edge; and in addition, attributes, that is, a number of nodes and a longest path of the graph, are added to additional molecular information provided by the global nodes.

5. The design method of a polymeric seawater desalination membrane based on a graph neural network according to claim 4, wherein a method for the initialization of the graph neural network in step S3 comprises:
when a main architecture of the graph neural network is initialized, generating an embedding layer of nodes based on neighbor nodes, wherein the embedding layer is in the following form:
$$h {v}^{k} = s ( Wk {\sum }_{u , N ( v )} \frac{h {u}^{k - 1}}{\vert N ( v ) \vert} + Bk {h}_{v}^{k - 1} ) ,$$
wherein v represents an index of an atomic node in the graph, k represents a number of current convolutional layers, N(v) represents all neighbors of the node v, σ is an activation function LeakyReLU, W and B are model parameters to be learned, edges have independent parameters and similar operations are performed, and h represents a weight and deviation of each node in this layer of the graph neural network;
providing a fully connected layer after the embedding layer to output a result; and
initializing a weight and deviation of the graph neural network to obtain an initial input of the graph neural network: hᵥ⁰=Xᵥ,
wherein X represents an input matrix of this layer of the graph neural network, comprising related attributes of the atoms of the desalination membrane polymer.

6. The design method of a polymeric seawater desalination membrane based on a graph neural network according to claim 5, wherein the training of the graph neural network in step S4 comprises the following substeps:
S4-1: performing normalization and data enhancement processing on initial input molecular information input by the graph neural network to make the initial input molecular information easy to converge and improve accuracy;
S4-2: constructing an embedding layer with the graph neural network as a main body, calculating a linear combination value of an input and a weight for each layer, and then applying the activation function to the linear combination value, which has different model parameters for nodes and edges;
S4-3: performing aggregation operation on nodes and edges in each layer, and aggregating weights of neighbor nodes and global nodes into nodes; and
S4-4: finally training by using the fully connected layer to calculate a loss function, and outputting the design model of the polymeric seawater desalination membrane.

7. The design method of a polymeric seawater desalination membrane based on a graph neural network according to claim 6, wherein a standard for evaluating seawater desalination membrane samples by using the design model of the polymeric seawater desalination membrane output in step S4-4, that is, a salt rejection rate, has the following expression:
salt rejection rate=1−(sodium ion+chloride ion passed)/(total sodium ion+chloride ion).

8. The design method of a polymeric seawater desalination membrane based on a graph neural network according to claim 6, wherein the design model of the polymeric seawater desalination membrane uses the graph neural network to learn graph structure data, extract and explore features and patterns in a molecular structure graph, and integrate graph convolution and an attention mechanism.

9. A computer device, comprising a memory, a processor, and a program stored in the processor and executable on the processor, wherein when the processor executes the program, the design method of a polymeric seawater desalination membrane based on a graph neural network according to claim 1 is implemented.

10. A computer-readable storage medium, which stores a computer program, wherein when the computer program is executed by a processor, the design method of a polymeric seawater desalination membrane based on a graph neural network according to claim 1 is implemented.

11. The computer device according to claim 9, wherein in step S1, data of the knowledge graph is screened and cleaned before the molecular structure information data set is constructed, the data of the knowledge graph comprises electronegativity and oxidation of molecules and groups, and synthesis difficulty and valence electrons of compounds, and a method for the data screening and cleaning of the knowledge graph comprises: matching in the literature and the database by using efficient string search and replacement algorithm flashtext, screening related data, and cleaning data with obvious errors and problems with reference to related chemical characteristics and a comparison with data of the same type.

12. The computer device according to claim 9, wherein in step S2, a method for the characterization processing for generating the adjacency matrix comprises: transforming the obtained molecular structural formula into a simplified molecular input line entry specification (SMILES) by using chemdraw software, and then obtaining the adjacency matrix of the molecule by using a GetAdjacencyMatrix method of an rdkit library.

13. The computer device according to claim 9, wherein in step S3, when the atoms are used as nodes of the graph, a serial number of each atom, a number and serial numbers of atoms adjacent to the atom, and related attributes of the atom are input, so that the atom is used as a node; when the molecular bonds are used as edges, a serial number and weight of each molecular bond and related attributes of the molecular bond are input, so that the molecular bond is used as an edge; and in addition, attributes, that is, a number of nodes and a longest path of the graph, are added to additional molecular information provided by the global nodes.

14. The computer device according to claim 13, wherein a method for the initialization of the graph neural network in step S3 comprises:
when a main architecture of the graph neural network is initialized, generating an embedding layer of nodes based on neighbor nodes, wherein the embedding layer is in the following form:
$$h {v}^{k} = s ( Wk {\sum }_{u , N ( v )} \frac{h {u}^{k - 1}}{\vert N ( v ) \vert} + Bk {h}_{v}^{k - 1} ) ,$$
wherein v represents an index of an atomic node in the graph, k represents a number of current convolutional layers, N(v) represents all neighbors of the node v, σ is an activation function LeakyReLU, W and B are model parameters to be learned, edges have independent parameters and similar operations are performed, and h represents a weight and deviation of each node in this layer of the graph neural network;
providing a fully connected layer after the embedding layer to output a result; and
initializing a weight and deviation of the graph neural network to obtain an initial input of the graph neural network: hᵥ⁰=Xᵥ,
wherein X represents an input matrix of this layer of the graph neural network, comprising related attributes of the atoms of the desalination membrane polymer.

15. The computer device according to claim 14, wherein the training of the graph neural network in step S4 comprises the following substeps:
S4-1: performing normalization and data enhancement processing on initial input molecular information input by the graph neural network to make the initial input molecular information easy to converge and improve accuracy;
S4-2: constructing an embedding layer with the graph neural network as a main body, calculating a linear combination value of an input and a weight for each layer, and then applying the activation function to the linear combination value, which has different model parameters for nodes and edges;
S4-3: performing aggregation operation on nodes and edges in each layer, and aggregating weights of neighbor nodes and global nodes into nodes; and
S4-4: finally training by using the fully connected layer to calculate a loss function, and outputting the design model of the polymeric seawater desalination membrane.

16. The computer device according to claim 15, wherein a standard for evaluating seawater desalination membrane samples by using the design model of the polymeric seawater desalination membrane output in step S4-4, that is, a salt rejection rate, has the following expression:
salt rejection rate=1−(sodium ion+chloride ion passed)/(total sodium ion+chloride ion).

17. The computer device according to claim 15, wherein the design model of the polymeric seawater desalination membrane uses the graph neural network to learn graph structure data, extract and explore features and patterns in a molecular structure graph, and integrate graph convolution and an attention mechanism.

18. The computer-readable storage medium according to claim 10, wherein in step S1, data of the knowledge graph is screened and cleaned before the molecular structure information data set is constructed, the data of the knowledge graph comprises electronegativity and oxidation of molecules and groups, and synthesis difficulty and valence electrons of compounds, and a method for the data screening and cleaning of the knowledge graph comprises: matching in the literature and the database by using efficient string search and replacement algorithm flashtext, screening related data, and cleaning data with obvious errors and problems with reference to related chemical characteristics and a comparison with data of the same type.

19. The computer-readable storage medium according to claim 10, wherein in step S2, a method for the characterization processing for generating the adjacency matrix comprises: transforming the obtained molecular structural formula into a simplified molecular input line entry specification (SMILES) by using chemdraw software, and then obtaining the adjacency matrix of the molecule by using a GetAdjacencyMatrix method of an rdkit library.

20. The computer-readable storage medium according to claim 10, wherein in step S3, when the atoms are used as nodes of the graph, a serial number of each atom, a number and serial numbers of atoms adjacent to the atom, and related attributes of the atom are input, so that the atom is used as a node; when the molecular bonds are used as edges, a serial number and weight of each molecular bond and related attributes of the molecular bond are input, so that the molecular bond is used as an edge; and in addition, attributes, that is, a number of nodes and a longest path of the graph, are added to additional molecular information provided by the global nodes.
